(19) United States
(12) Patent Application Publication
(10) Pub. No.: US 2023/0358668 A1
(43) Pub. Date: Nov. 9, 2023

(54) INSPECTION DEVICE AND INSPECTION METHOD

(71) Applicant: GIMER MEDICAL. Co. LTD., New Taipei City (TW)

(72) Inventor: Chi-Yu HUANG, New Taipei City (TW)

(73) Assignee: GIMER MEDICAL. Co. LTD., New Taipei City (TW)

(21) Appl. No.: 18/142,867

(22) Filed: May 3, 2023

(30) Foreign Application Priority Data
May 6, 2022 (CN) 202210486775.9

(51) Int. Cl. G01N 21/21; A61B 5/1459; A61B 5/1495

(52) U.S. Cl. CPC G01N 21/21; A61B 5/1459; A61B 5/1495; G01N 2201/127; G01N 2201/0636; G01N 2201/08; A61B 2562/0233; G02B 27/14

(57) ABSTRACT

An inspection device for inspecting an analyte in a liquid is provided, including a light source module, a first polarizing member, a second polarizing member, a sensing member, and a calibration assembly. The light source module provides a light, and the first polarizing member is disposed between the light source module and the second polarizing member. An accommodating space for accommodating the aforementioned liquid is formed between the first polarizing member and the second polarizing member. The second polarizing member is disposed between the first polarizing member and the sensing member. When the inspection device inspects the liquid, a portion of the light passes through the first polarizing member, the liquid, and the second polarizing member and reaches the sensing member, and another portion of the light enters the calibration assembly.

### CROSS REFERENCE TO RELATED APPLICATIONS

The present application claims priority of China Patent Application No. 202210486775.9, filed May 6, 2022, which is incorporated by reference herein in its entirety.

### BACKGROUND OF THE INVENTION

### Field of the Invention

The application relates in general to an inspection device, and in particular, to an inspection device for measuring an analyte in a liquid.

### Description of the Related Art

With the advancement of medical treatment, the human life span is getting longer, meaning that there are a greater number of patients with metabolic disorders. For example, diabetes is an important example of a metabolic disorder. Patients need to monitor their blood sugar levels, so as to prevent complications.

Currently, there are two common inspection methods, one of which involves dripping blood on a test paper. Another involves the insertion of a probe into the human body to measure the amount of glucose in the blood or another bodily fluid. However, the results of the aforementioned measurement methods can easily become inaccurate due to contamination, and the inspection methods are not very convenient to inspect, meaning that the required information cannot be obtained at all times. Therefore, how to address the aforementioned problem has become an important issue.

### BRIEF SUMMARY OF INVENTION

To address the deficiencies of conventional products, an embodiment of the disclosure provides an inspection device for inspecting an analyte in a liquid, including a light source module, a first polarizing member, a second polarizing member, a sensing member, and a calibration assembly. The light source module provides a light, and the first polarizing member is disposed between the light source module and the second polarizing member. An accommodating space for accommodating the aforementioned liquid is formed between the first polarizing member and the second polarizing member. The second polarizing member is disposed between the first polarizing member and the sensing member. When the inspection device inspects the liquid, a portion of the light passes through the first polarizing member, the liquid, and the second polarizing member and reaches the sensing member, and another portion of the light enters the calibration assembly.

In some embodiments, the calibration assembly comprises a light splitting member, a calibration polarizing member, and a calibration sensing member. The light splitting member is disposed between the first polarizing member and the accommodating space, and the calibration polarizing member is disposed between the light splitting member and the calibration sensing member. The polarizing angle of the calibration polarizing member is the same as the polarizing angle of the second polarizing member. In some embodiments, the calibration assembly further comprises a lock-in amplifier electrically connected to the calibration sensing member.

In some embodiments, the calibration assembly comprises a first light splitting member, a calibration polarizing member, at least one reflecting member, and a second light splitting member. The first light splitting member is disposed between the first polarizing member and the accommodating space, and the calibration polarizing member is disposed between the first light splitting member and the reflecting member. The second light splitting member is disposed between the second polarizing member and the sensing member, wherein the polarizing angle of the calibration polarizing member is the same as the polarizing angle of the second polarizing member. When the inspection device inspects the liquid, the first light splitting member guides a portion of the light to the calibration polarizing member. This portion of the light passes through the calibration polarizing member and is reflected to the second splitting member by the reflecting member, and the second splitting member guides this portion of the light to the sensing member. An additional accommodating space is disposed between the reflecting member and the second light splitting member, and the liquid does not enter the additional accommodating space.

In some embodiments, the calibration assembly comprises a first light splitting member, a calibration polarizing member, a second light splitting member, and a fiber member. The first light splitting member is disposed between the first polarizing member and the accommodating space. The second light splitting member is disposed between the second polarizing member and the sensing member. The polarizing angle of the calibration polarizing member is the same as the polarizing angle of the second polarizing member. The fiber member has a first end and a second end, wherein the first end faces the calibration polarizing member, the second end faces the second splitting member, and the calibration polarizing member is disposed between the first end and the first light splitting member.

In some embodiments, the calibration assembly comprises a light splitting member, a calibration polarizing member, and a fiber member. The light splitting member is disposed between the first polarizing member and the accommodating space. The polarizing angle of the calibration polarizing member is the same as the polarizing angle of the second polarizing member. The fiber member has a first end and a second end, wherein the first end faces the calibration polarizing member, the second end faces the sensing member, and the calibration polarizing member is disposed between the first end and the light splitting member. In some embodiments, the calibration assembly further comprises a first shutter and a second shutter. The first shutter is disposed between the light splitting member and the accommodating space, and the second shutter is disposed between the light splitting member and the calibration polarizing member.

In some embodiments, the calibration assembly comprises a light splitting member and a calibration sensing member. The light splitting member is disposed between the accommodating space and the second polarizing member, and the light splitting member guides a portion of the light to the calibration sensing member. In some embodiments, the calibration assembly further comprises a lock-in amplifier electrically connected to the calibration sensing member.

In some embodiments, the calibration assembly further comprises a calibration sensing member, and the second polarizing member is disposed between the first polarizing member and the calibration sensing member. The second polarizing member has a first section and a second section, the first section has the function of polarizing, the second section does not have the function of polarizing, and the sensing member and the calibration sensing member respectively correspond to the first section and the second section.

In some embodiments, the inspection device further comprises a lock-in amplifier, a first chiral mirror, and a second chiral mirror. The lock-in amplifier is electrically connected to the sensing member, the first chiral mirror is disposed between the first polarizing member and the accommodating space, and the second chiral mirror is disposed between the accommodating space and the second polarizing member.

In some embodiments, the light source module comprises a light source, and the calibration assembly is disposed in the light source module. The calibration assembly comprises a calibration polarizing member, a sensor, an automatic power control driver, and a polarization member. The polarization member is disposed between the light source and the calibration polarizing member, and the calibration polarizing member is disposed between the sensor and the polarization member. The polarizing angle of the polarization member is the same as the polarizing angle of the first polarizing member. The automatic power control driver is electrically connected to the sensor and the light source. The calibration polarizing member and the first polarizing member are disposed on different sides of the light source.

An embodiment of the invention further provides an inspection method, including: providing an inspection device; letting a liquid enter the accommodating space; using the light source module to provide a light, wherein a portion of the light passes through the first polarizing member, the liquid, and the second polarizing member and reaches the sensing member, and another portion of the light enters the calibration assembly; and transferring measuring data from the inspection device to a reading device.

### BRIEF DESCRIPTION OF DRAWINGS

The invention can be more fully understood by reading the subsequent detailed description and examples with references made to the accompanying drawings, wherein:

FIG. 1A is a schematic diagram representing that an inspection device and a reading device transmit signal in a wireless manner according to an embodiment of the invention;

FIG. 1B is a schematic diagram representing that an inspection device and a reading device transmit signal in a wireless manner via a transmitter according to an embodiment of the invention;

FIG. 2A is a schematic diagram of the inspection device according to an embodiment of the invention;

FIG. 2B is a schematic diagram of a hollow carrier configured to accommodate the inspection device according to an embodiment of the invention;

FIG. 3 is a schematic diagram of an inspection device according to another embodiment of the invention;

FIG. 4 is a schematic diagram of an inspection device according to another embodiment of the invention;

FIG. 5 is a schematic diagram of an inspection device according to another embodiment of the invention;

FIG. 6 is a schematic diagram of an inspection device according to another embodiment of the invention;

FIG. 7 is a schematic diagram of an inspection device according to another embodiment of the invention;

FIG. 8 is a schematic diagram of an inspection device according to another embodiment of the invention;

FIG. 9A is a schematic diagram of an inspection device according to another embodiment of the invention;

FIG. 9B is a schematic diagram of a light source module and a calibration assembly in FIG. 9A;

FIG. 10 is a flow chart of an inspection method according to an embodiment of the invention;

FIG. 11A is a schematic diagram of an inspection device according to another embodiment of the invention;

FIG. 11B is a cross-sectional view taken along the line M-M in FIG. 11A;

FIG. 11C is a cross-sectional view taken along the line N-N in FIG. 11A; and

FIG. 11D is a partial enlarged view of FIG. 11B.

### DETAILED DESCRIPTION OF INVENTION

The making and using of the embodiments of the inspection device are discussed in detail below. It should be appreciated, however, that the embodiments provide many applicable inventive concepts that can be embodied in a wide variety of specific contexts. The specific embodiments discussed are merely illustrative of specific ways to make and use the embodiments, and do not limit the scope of the disclosure.

Unless defined otherwise, all technical and scientific terms used herein have the same meaning as commonly understood by one of ordinary skill in the art to which this invention belongs. It should be appreciated that each term, which is defined in a commonly used dictionary, should be interpreted as having a meaning conforming to the relative skills and the background or the context of the present disclosure, and should not be interpreted in an idealized or overly formal manner unless defined otherwise.

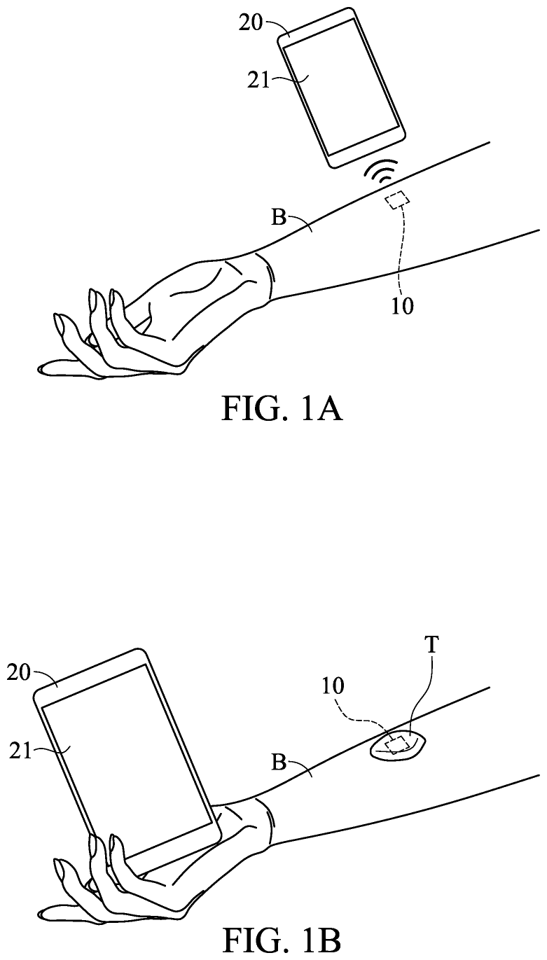

Referring to FIG. 1A, an inspection device 10 in an embodiment can be implanted into a subcutaneous tissue of a human body B to continuously inspect the concentration of an analyte (such as glucose, urate crystal, lactic acid, etc.) in a bodily fluid of the human body B, or the presence or absence of the analyte in a bodily fluid of the human body B. The continuous measurement can be defined as a measurement every 0.5, 1, 3, 5, 10, 15, 20, 30, 40, 50, 60, 90, 120, or 180 minutes, or an uninterrupted measurement.

When the user desires to obtain the measuring result of the analyte in the bodily fluid, a reading device 20 can be attached to or close to the skin surface of the human body B where the inspection device 10 is embedded. At that time, the inspection device 10 can transmit the inspected data to the reading device 20 in a wireless manner, and the reading device 20 can display the aforementioned data on its display panel 21. Therefore, it is convenient for the user to obtain the information of the analyte, and further understand his/her own body conditions.

As shown in FIG. 1B, in some embodiments, a transmitter T can be attached to the skin surface of the human body B where the inspection device 10 is embedded, so that the inspection device 10 can transmit the inspected data to the transmitter T in a wireless manner, and the transmitter T can subsequently transmit the data to the reading device 20. Therefore, the range that the reading device 20 can receive the data is increased.

The reading device 20 can further include a wireless power supply. When the reading device 20 is attached to or close to the skin surface of the human body B where the inspection device 10 is embedded, it can provide power to the inspection device 10 by the wireless power supply. After the inspection device 10 receives power, it can be operated to measure and transmit the data. In some embodiments, the inspection device 10 can include battery, so that it can be operated at all times to measure the analyte. In an embodiment of the invention, the reading device 20 can be a smartphone, a tablet computer, or other portable electronic device.

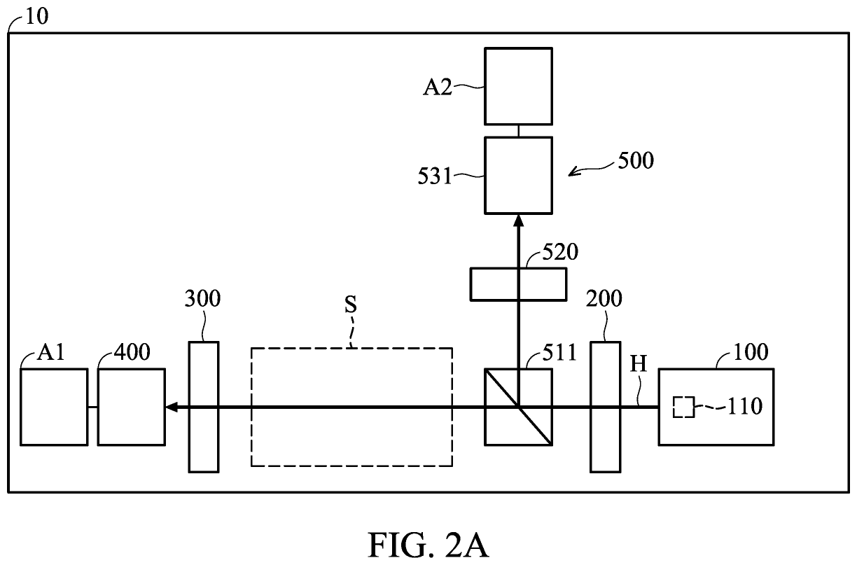

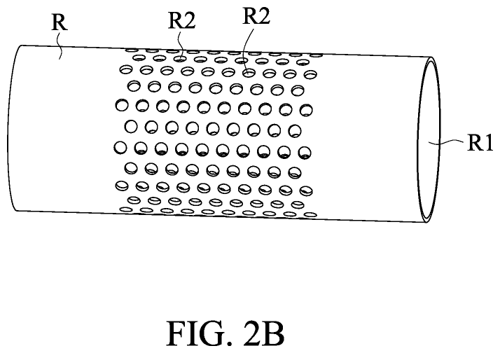

It should be noted that, the inspection device 10 is embedded into the human body B to take an example in this embodiment, but it is not limited thereto. The inspection device 10 can also measure the analyte in the liquid when it is not embedded into the human body B (i.e. outside the human body). For example, the analyte (the tissue fluid, the blood, or other body fluid) can be taken firstly, and then the analyte can be put into the inspection device 10 to inspect. Furthermore, the non-implantable inspection device can be disposed on the skin surface of the human body, such as to be integrated with the transmitter T shown in FIG. 1B. The non-implantable inspection device can include a hose needle to pierce into the skin (such as the arm or the abdomen), so that the analyte can be taken to the accommodating space S shown in FIG. 2B to inspect, and the cavity portions R2 can be omitted. The accommodating space S and cavity portions R2 are discussed in detail below.

FIG. 2A is a schematic diagram of the aforementioned inspection device 10. As shown in FIG. 2A, the inspection device 10 primarily includes a light source module 100, a first polarizing member 200, a second polarizing member 300, a sensing member 400, a calibration assembly 500, and a processing module A1.

The light source module 100 includes one or more light sources 110. For example, the light source 110 can be a light-emitting diode (LED) or a laser diode, and disposed on a printed circuit board or a flexible board. The light source 110 can emit a light with a wavelength between the infrared light and the ultraviolet light, for example, the light with the wavelength ranged in 300 nm-2000 nm. Since the laser diode is coherent and highly directional, it can be a preferable choice. In an embodiment of the invention, the light source 110 can emit a light with the frequency ranged in 100 Hz-100000 Hz. Preferably, the light source 110 can emit a light with the frequency of 1000 Hz, but it is not limited thereto.

The first polarizing member 200 can be a polarizer, and can be disposed between the light source module 100 and the second polarizing member 300. The first polarizing member 200 is configured to purify the polarizing angle of the light from the light source module 100. In an embodiment of the invention, the extinction ratio of the first polarizing member 200 is larger than 10000, but it is not limited thereto.

The second polarizing member 300 can be a polarizer, and can be disposed between the first polarizing member 200 and the sensing member 400. The polarizing angle of the second polarizing member 300 is different from the polarizing angle of the first polarizing member 200. In this embodiment, the polarizing angles of the second polarizing member 300 and the first polarizing member 200 are orthogonal. For example, the polarizing angle of the first polarizing member 200 can be zero degree, and the polarizing angle of the second polarizing member 300 can be ninety degrees. In an embodiment of the invention, the extinction ratio of the second polarizing member 300 is larger than 10000, but it is not limited thereto.

An accommodating space S that the liquid (such as the body fluid of the human body) is enable to enter is formed between the first polarizing member 200 and the second polarizing member 300. For example, as shown in FIG. 2B, the inspection device 10 can be disposed in a hollow carrier R, and the hollow carrier R can have an inner space R1 and one or more cavity portions R2 communicated with the inner space R1. The position of the inner space R1 corresponding to the cavity portions R2 is the accommodating space S. The light source module 100, the first polarizing member 200, the second polarizing member 300, the sensing member 400, the calibration assembly 500, and the processing module A1 can be disposed in the position of the inner space R1 where does not corresponded to the cavity portions R2.

When the liquid enters the accommodating space S, the light H from the light source module 100 can pass through the first polarizing member 200, the liquid, and the second first polarizing member 300 in sequence and then reach the sensing member 400. If the liquid has chiral material (i.e. the analyte), the polarizing direction of the light H is rotated. After the sensing member 400 receives the light H that is rotated by chiral material, the sensing member 400 can transfer the light signal to the electric signal and transmit the electric signal to the processing module A1. The processing module A1 can obtain the concentration of the analyte, the presence or absence of the analyte, or whether the amount of the analyte is greater than a threshold by inspecting the optical rotation and/or the light absorption of the light. In an embodiment of the invention, the sensing member 400 can be a photodiode sensor, a charge coupled device (CCD) sensor, a complementary metal oxide semiconductor (CMOS) sensor, etc., but it is not limited thereto. The processing module A1 can include a low- and high-pass filter, a band elimination filter, a differential amplifier circuit, a lock-in amplifier circuit, and/or a microprocessor, but it is not limited thereto.

For example, the concentration of the analyte can be obtained according to the following formula:

$$C = \frac{θ}{{[ α ]}_{λ . T}^{p H} \cdot L}$$

wherein C is the concentration of the analyte (g/100 mL), θ is the specific rotation inspected by the processing module A1, [a]_{λ, T}^{pH} is the specific rotation (for example, the specific rotation of glucose is +52.7° dm⁻¹(g/ml)⁻¹), and L is the light path (decimeter). According to the aforementioned formula, it can be noted that the concentration of the analyte is proportional to the specific rotation. The specific rotation and the light path of the analyte are already known, so that a standard curve line of the analyte with the known concentration can be established, and the concentration of the analyte or the presence or absence of the analyte can be calculated.

Referring to FIG. 2A, in this embodiment, the calibration assembly 500 includes a light splitting member 511, a calibration polarizing member 520, a calibration sensing member 531, and a processing module A2. The light splitting member 511 is disposed between the first polarizing member 200 and the accommodating space S, the calibration polarizing member 520 is disposed between the light splitting member 511 and the calibration sensing member 531, and the accommodating space S and the calibration polarizing member 520 are disposed on different sides of the light splitting member 511. The light splitting member 511 can be a beam splitter. Therefore, when the light H from the light source module 100 enters the light splitting member 511, a portion of the light H passes through the light splitting member 511 and moves toward the accommodating space S, and another portion of the light H is reflected by the light splitting member 511 to the calibration polarizing member 520. In an embodiment of the invention, the calibration sensing member 531 can be a photodiode sensor, a charge coupled device (CCD) sensor, a complementary metal oxide semiconductor (CMOS) sensor, etc., but it is not limited thereto.

The polarizing angle of the calibration polarizing member 520 can be the same as the polarizing angle of the second polarizing member 300, and the light H reflected by the light splitting member 511 can pass through the calibration polarizing member 520 and reach the calibration sensing member 531. After the calibration sensing member 531 receives the light H, it can transfer the light signal to the electric signal and transmit the electric signal to the processing module A2. The composition of the processing module A2 can be the same as that of the processing module A1. Since the moving path of the light H from the light splitting member 511 to the calibration sensing member 531 does not pass through the liquid, the processing module A2 can measure the result that is not influenced by the analyte in the liquid. When the reading device 20 is attached or close to the skin surface of the human body B where the inspection device 10 is embedded, the processing module A1 and the processing module A2 can simultaneously transfer theirs measuring results to the reading device 20, and the reading device 20 can calibrate the data measured by the processing module A1 according to the measuring result of the processing module A2. Thus, the measuring accuracy of the inspection device 10 can be enhanced.

In some embodiments, the distance between the light splitting member 511 and the sensing member 400 is substantially the same as the distance between the light splitting member 511 and the calibration sensing member 531. The travel distances of the light H to the sensing member 400 and the calibration sensing member 531 are the same, so that the calibration effect can be further enhanced. In some embodiments, the splitting member 511 can be a rotatable mirror, and it can rotate to different angle to adjust the light H to move in the direction toward the accommodating space S or in the direction toward the calibration polarizing member 520.

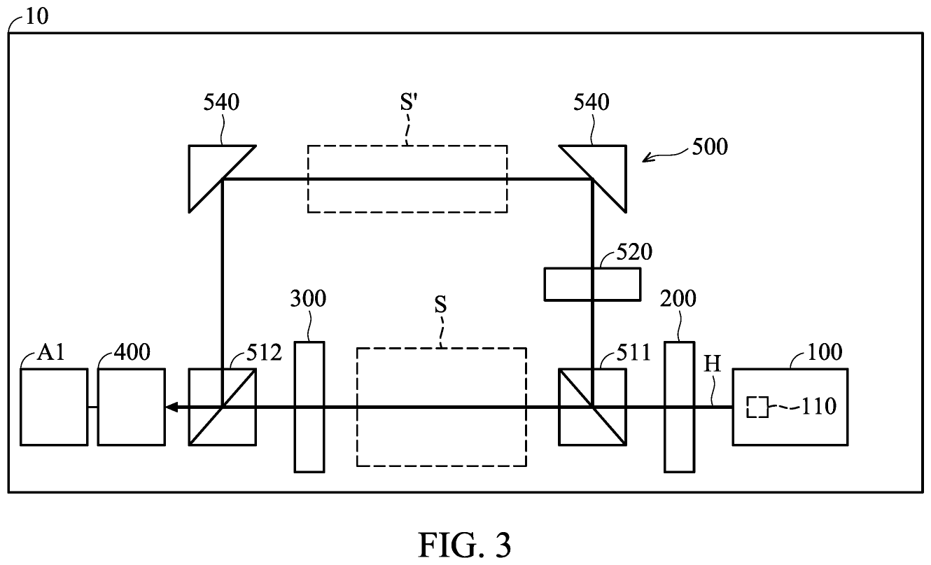

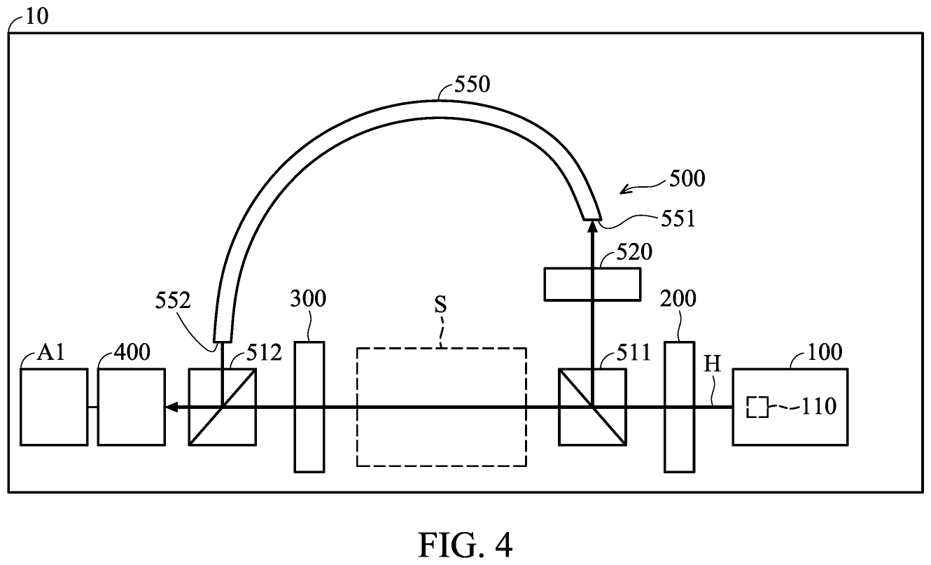

Referring to FIG. 3, in another embodiment, the inspection device 10 primarily includes a light source module 100, a first polarizing member 200, a second polarizing member 300, a sensing member 400, a calibration assembly 500, and a processing module A1. The light source module 100, the first polarizing member 200, the second polarizing member 300, and the sensing member 400 are the same as that shown in the embodiment of FIG. 2, so that the features thereof are not repeated in the interest of brevity.

In this embodiment, the calibration assembly 500 includes a light splitting member 511 (a first light splitting member), a calibration polarizing member 520, at least one reflecting member 540, and a light splitting member 512 (a second light splitting member). The light splitting member 511 is disposed between the first polarizing member 200 and the accommodating space S, the calibration polarizing member 520 is disposed between the light splitting member 511 and the reflecting member 540, and the accommodating space S and the calibration polarizing member 520 are disposed on different sides of the light splitting member 511. The light splitting member 511 can be a beam splitter. Therefore, when the light H from the light source module 100 enters the light splitting member 511, a portion of the light H passes through the light splitting member 511 and moves toward the accommodating space S, and another portion of the light H is reflected by the light splitting member 511 to the calibration polarizing member 520. The polarizing angle of the calibration polarizing member 520 is substantially the same as the polarizing angle of the second polarizing member 300.

The light splitting member 512 is disposed between the second polarizing member 300 and the sensing member 400. The light splitting member 512 can also be a beam splitter. The light H moving toward the accommodating space S can pass through the light splitting member 512 and reach the sensing member 400. The light H reflected by the light splitting member 511 can pass through the calibration polarizing member 520, and then can be reflected by the reflecting member 540 to the light splitting member 512. The light splitting member 512 can reflect this portion of light H to the sensing member 400.

It should be noted that, when the user uses the inspection device 10 to inspect the liquid, the liquid should not enter the space between the reflecting member 540 and the light splitting member 512 (such as the accommodating space S′). Thus, the sensing member 400 can receive the light H rotated by chiral material and the light H that is not rotated by chiral material, and transfer both from the light signal to the electric signal and transmit them to the processing module A1. Therefore, the processing module A1 can obtain the stability of light H from the light source module 100, and calibrate the measuring result of the analyte. The measuring accuracy of the inspection device 10 can be enhanced.

In some embodiments, the splitting member 511 and/or the light splitting member 512 can be a rotatable mirror configured to adjust the moving direction of the light H.

Since the inspection device 10 in this embodiment includes the single sensing member 400, the cost of manufactory can be reduced and the influence of the difference response curve in different sensing members can be removed compared with the embodiment shown in FIG. 2.

Referring to FIG. 4, in another embodiment, the inspection device 10 primarily includes a light source module 100, a first polarizing member 200, a second polarizing member 300, a sensing member 400, a calibration assembly 500, and a processing module A1. The light source module 100, the first polarizing member 200, the second polarizing member 300, and the sensing member 400 are the same as that shown in the embodiment of FIG. 2, so that the features thereof are not repeated in the interest of brevity.

In this embodiment, the calibration assembly 500 includes a light splitting member 511 (a first light splitting member), a calibration polarizing member 520, a fiber member 550, and a light splitting member 512 (a second light splitting member). The light splitting member 511 is disposed between the first polarizing member 200 and the accommodating space S, and the light splitting member 511 can be a beam splitter. When the light H from the light source module 100 enters the light splitting member 511, a portion of the light H passes through the light splitting member 511 and moves toward the accommodating space S, and another portion of the light H is reflected by the light splitting member 511 to the calibration polarizing member 520. The polarizing angle of the calibration polarizing member 520 is substantially the same as the polarizing angle of the second polarizing member 300.

The light splitting member 512 is disposed between the second polarizing member 300 and the sensing member 400. The light splitting member 512 can also be a beam splitter. The fiber member 550 has a first end 551 and the second end 552. The first end 551 faces the calibration polarizing member 520, and the second end 552 faces the light splitting member 512.

The light H moving toward the accommodating space S can pass through the light splitting member 512 and reach the sensing member 400. The light H reflected by the light splitting member 511 can pass through the calibration polarizing member 520, and then enter the fiber member 550 via the first end 551 and leave the fiber member 552 via the second end 552. The light splitting member 512 can reflect this portion of light H to the sensing member 400. Thus, when the user uses the inspection device 10 to inspect the liquid, the sensing member 400 can receive the light H rotated by chiral material and the light H that is not rotated by chiral material, and transfer both from the light signal to the electric signal and transmit them to the processing module A1. Therefore, the processing module A1 can obtain the stability of light H from the light source module 100, and calibrate the measuring result of the analyte. The measuring accuracy of the inspection device 10 can be enhanced.

In some embodiments, the splitting member 511 and/or the light splitting member 512 can be a rotatable mirror configured to adjust the moving direction of the light H.

In this embodiment, there is no need to arrange a space in the inspection device 10 for the light H to move (such as the accommodating space S′), so that the miniaturization of the inspection device 10 can be facilitated compared with the embodiment of FIG. 3.

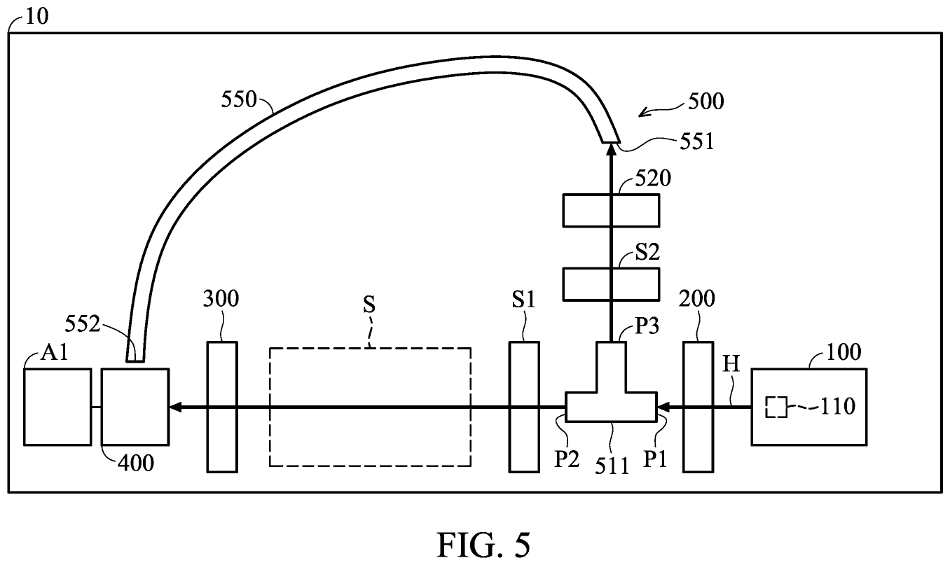

Referring to FIG. 5, in another embodiment, the inspection device 10 primarily includes a light source module 100, a first polarizing member 200, a second polarizing member 300, a sensing member 400, a calibration assembly 500, and a processing module A1. The light source module 100, the first polarizing member 200, the second polarizing member 300, and the sensing member 400 are the same as that shown in the embodiment of FIG. 2, so that the features thereof are not repeated in the interest of brevity.

In this embodiment, the calibration assembly 500 includes a light splitting member 511, a calibration polarizing member 520, a fiber member 550, a first shutter S1, and a second shutter S2. The light splitting member 511 is disposed between the first polarizing member 200 and the accommodating space S, and the light splitting member 511 can be a fiber coupler with one light-entering port P1 and two light-emitting ports P2 and P3. The first shutter S1 is disposed between the light-emitting port P2 and the accommodating space S, and the second shutter S2 is disposed between the light-emitting port P3 and the calibration polarizing member 520.

The fiber member 550 has a first end 551 and the second end 552. The first end 551 faces the calibration polarizing member 520, and the second end 552 faces the sensing member 400. The polarizing angle of the calibration polarizing member 520 is substantially the same as the polarizing angle of the second polarizing member 300.

The light H from the light source module 100 can enter the light splitting member 511 via the light-entering port P1, and leave the light splitting member 511 via the light-emitting ports P2 and P3. The light H leaving from the light-emitting port P2 can pass through the first shutter S1, the accommodating space S, and the second polarizing member 300 in sequence and reach the sensing member 400. The light H leaving from the light-emitting port P3 can pass through the second shutter S2 and the calibration polarizing member 520, then enter the fiber member 550 via the first end 551 and leave the fiber member 550 via the second end 552, and reach the sensing member 400. Thus, when the user uses the inspection device 10 to inspect the liquid, the sensing member 400 can receive the light H rotated by chiral material and the light H that is not rotated by chiral material, and transfer both from the light signal to the electric signal and transmit them to the processing module A1. Therefore, the processing module A1 can obtain the stability of light H from the light source module 100, and calibrate the measuring result of the analyte. The measuring accuracy of the inspection device 10 can be enhanced.

The first shutter S1 and the second shutter S2 can be opened or closed, so that the sensing member 400 does not need to measure the light H in different path simultaneously.

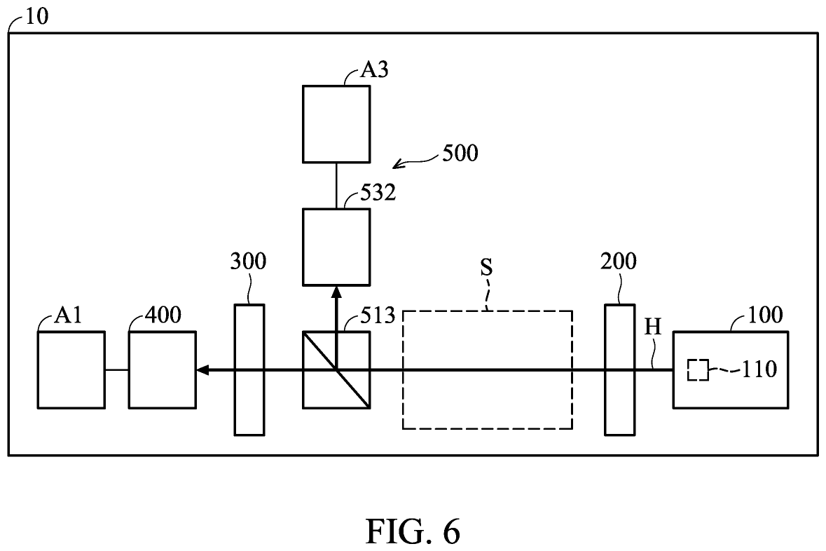

Referring to FIG. 6, in another embodiment, the inspection device 10 primarily includes a light source module 100, a first polarizing member 200, a second polarizing member 300, a sensing member 400, a calibration assembly 500, and a processing module A1. The light source module 100, the first polarizing member 200, the second polarizing member 300, the sensing member 400, and the processing module A1 are the same as that shown in the embodiment of FIG. 2, so that the features thereof are not repeated in the interest of brevity.

In this embodiment, the calibration assembly 500 includes a light splitting member 513, a calibration sensing member 532, and a processing module A3. The light splitting member 513 is disposed between the accommodating space S and the second polarizing member 300, and the second polarizing member 300 and the calibration sensing member 532 are disposed on different sides of the light splitting member 513. The light splitting member 513 can be a beam splitter. When the light H enters the light splitting member 513, a portion of the light H passes through the light splitting member 513 and moves toward the second polarizing member 300, and another portion of the light H is reflected by the light splitting member 513 to the calibration sensing member 532. When the calibration sensing member 532 receives the light H, it can transfer the light signal to the electric signal and transmit the electric signal to the processing module A3. In an embodiment of the invention, the calibration sensing member 532 can be a photodiode sensor, a charge coupled device (CCD) sensor, a complementary metal oxide semiconductor (CMOS) sensor, etc., but it is not limited thereto. The processing module A3 can include a low- and high-pass filter, a band elimination filter, a differential amplifier circuit, a lock-in amplifier circuit, and/or a microprocessor, but it is not limited thereto.

It should be noted that, there is no polarizing member disposed between the light splitting member 513 and the calibration sensing member 532. Thus, when the reading device 20 is attached to or close to the skin surface of the human body B where the inspection device 10 is embedded, the processing module A1 and the processing module A3 can transmit the measuring results to the reading device 20, and the reading device 20 can determine that whether the data measured from the processing module A1 is deviated due to the degree of the light absorption of the liquid according to the measuring result of the processing module A3, and calibrate it. The measuring accuracy of the inspection device 10 can be enhanced.

When the processing module A1 and the processing module A3 include the lock-in amplifier, they can enlarge the small signal and remove the noise, so that the measuring result can be facilitated to obtain. In an embodiment of the invention, the processing module A1 and the processing module A3 include the lock-in amplifier with the frequency in 1000 Hz, but it is not limited thereto.

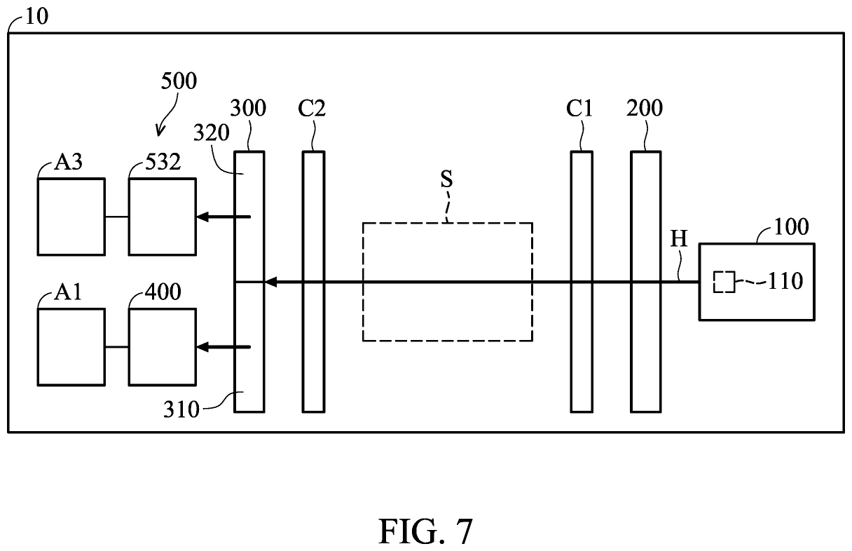

Referring to FIG. 7, in another embodiment, the inspection device 10 primarily includes a light source module 100, a first polarizing member 200, a second polarizing member 300, a sensing member 400, a calibration assembly 500, and a processing module A1. The light source module 100, the first polarizing member 200, the sensing member 400, and the processing module A1 are the same as that shown in the embodiment of FIG. 2, so that the features thereof are not repeated in the interest of brevity.

The second polarizing member 300 has a first section 310 and a second section 320, wherein the first section 310 has the function of polarizing, and the second section 320 does not have the function of polarizing. In this embodiment, the polarizing angles of the first section 310 and the first polarizing member 200 are substantially orthogonal. The calibration assembly 500 includes a calibration sensing member 532 and a processing module A3, and positions of the sensing member 400 and the calibration sensing member 532 are respectively corresponded to the first section 310 and the second section 320 of the second polarizing member 300. Therefore, after the light H from the light source module 100 passes through the first polarizing member 200 and the accommodating space S, a portion of the light H passes through the first section 310 of the polarizing member 300 and reaches the sensing member 400, and another portion of the light H passes through the second section 320 of the polarizing member 300 and reaches the calibration sensing member 532. When the calibration sensing member 532 receives the light H, it can transfer the light signal to the electric signal and transmit the electric signal to the processing module A3.

When the reading device 20 is attached to or close to the skin surface of the human body B where the inspection device 10 is embedded, the processing module A1 and the processing module A3 can transmit the measuring results to the reading device 20, and the reading device 20 can determine that whether the data measured from the processing module A1 is deviated due to the degree of the light absorption of the liquid according to the measuring result of the processing module A3, and calibrate it. The measuring accuracy of the inspection device 10 can be enhanced.

Since the light source 100, the first polarizing member 200, the second polarizing member 300, the sensing member 400, the calibration assembly 500 are substantially arranged along a straight line in this embodiment, the whole volume of the inspection device 10 can be effectively reduced, and the miniaturization of the inspection device 10 can be facilitated.

When the processing module A1 and the processing module A3 include the lock-in amplifier, they can enlarge the small signal and remove the noise, so that the measuring result can be facilitated to obtain. Moreover, in this embodiment, the inspection device 10 further includes a first chiral mirror C1 and a second chiral mirror C2 respectively disposed on opposite sides of the accommodating space S. Owing to the first chiral mirror C1 and the second chiral mirror C2, the light path can be increased, and the chiral signal can be enlarged. It should be noted that, the first chiral mirror C1 and the second chiral mirror C2 can also be applied in other embodiments in the invention.

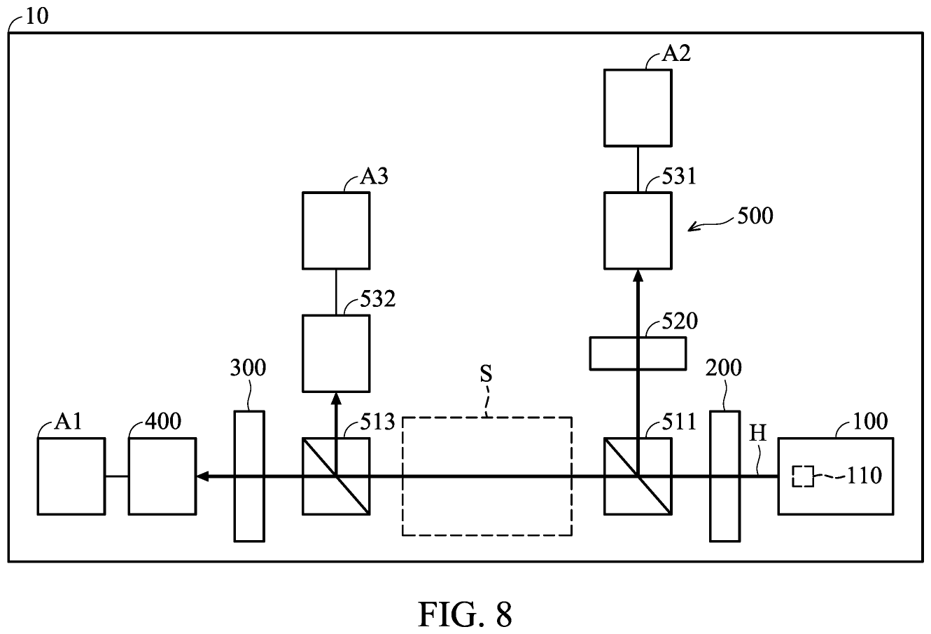

Referring to FIG. 8, in another embodiment, the inspection device 10 primarily includes a light source module 100, a first polarizing member 200, a second polarizing member 300, a sensing member 400, a calibration assembly 500, and a processing module A1. The light source module 100, the first polarizing member 200, the second polarizing member 300, the sensing member 400, and the processing module A1 are the same as that shown in the embodiment of FIG. 2, so that the features thereof are not repeated in the interest of brevity.

In this embodiment, the calibration assembly 500 includes a light splitting member 511, a calibration polarizing member 520, a calibration sensing member 531, a light splitting member 513, a calibration sensing member 532, a processing module A2, and a processing module A3. The structures and the arrangements of the light splitting member 511, the calibration polarizing member 520, the calibration sensing member 531, and the processing module A2 are the same as that shown in the embodiment of FIG. 2, and the structures and the arrangements of the light splitting member 513, the calibration sensing member 532, and the processing module A3 are the same as that shown in the embodiment of FIG. 6. Therefore, the measuring accuracy of the inspection device 10 in this embodiment can be further enhanced.

When the processing modules A1, A2, and A3 include the lock-in amplifier, they can enlarge the small signal and remove the noise, so that the measuring result can be facilitated to obtain. In an embodiment of the invention, the processing module A3 includes the lock-in amplifier with the frequency in 1000 Hz, but it is not limited thereto.

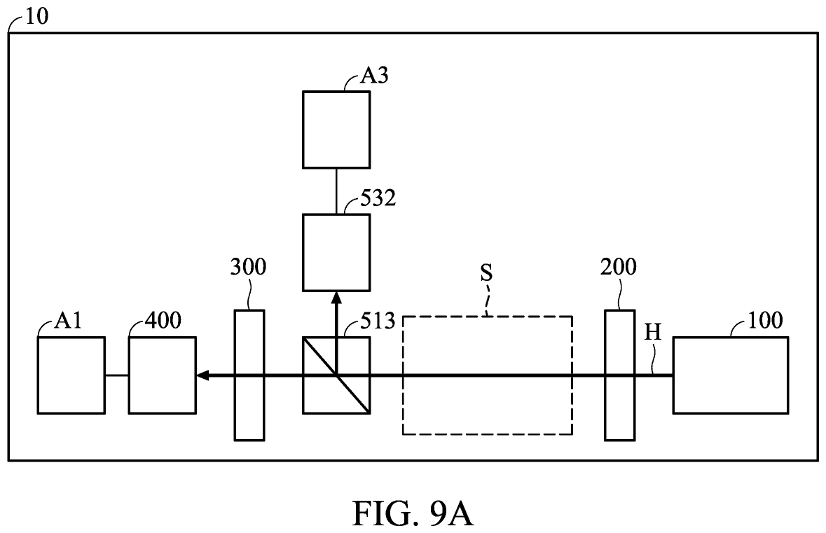

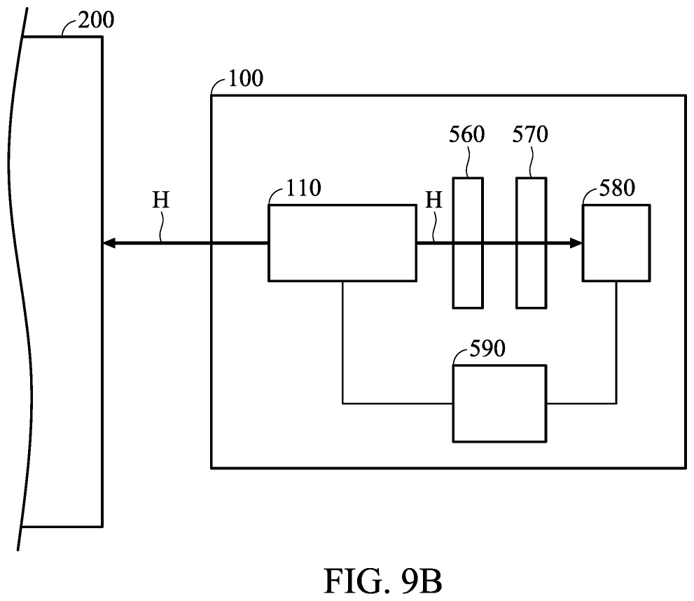

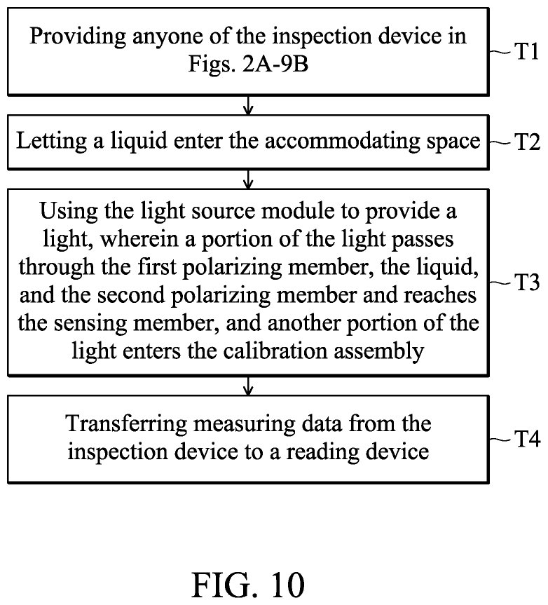

Referring to FIG. 9A and FIG. 9B, in another embodiment, the inspection device 10 primarily includes a light source module 100, a first polarizing member 200, a second polarizing member 300, a sensing member 400, a calibration assembly 500, and a processing module A1. The first polarizing member 200, the second polarizing member 300, the sensing member 400, and the processing module A1 are the same as that shown in the embodiment of FIG. 2, so that the features thereof are not repeated in the interest of brevity.

The calibration assembly 500 includes a light splitting member 513, a calibration sensing member 532, a processing module A3, a polarization member 560, a calibration polarizing member 570, a sensor 580, and an automatic power control driver 590. The structures and the arrangements of the light splitting member 513, the calibration sensing member 532, and the processing module A3 are the same as that shown in the embodiment of FIG. 6, so that the measuring result of the processing module A3 can be used to determine that whether the measurement of sensing member 400 is deviated due to the degree of the light absorption of the liquid, and calibrate it.

The polarization member 560, the calibration polarizing member 570, the sensor 580, and the automatic power control driver 590 are disposed in the light source module 100. The polarization member 560 is disposed between the calibration polarizing member 570 and the light source 110 of the light source module 100. The calibration polarizing member 570 is disposed between the sensor 580 and the polarization member 560. The automatic power control driver 590 is electrically connected to the sensor 580 and the light source 110. The first polarizing member 200 and the calibration polarizing member 570 are disposed on different sides of the light source 110 (in this embodiment, they are disposed on opposite sides). The polarizing angle of the polarization member 560 is the same as the polarizing angle of the first polarizing member 200, and the polarizing angle of the calibration polarizing member 570 is the same as the polarizing angle of the second polarizing member 300. In an embodiment of the invention, the sensor 580 can be a photodiode sensor, a charge coupled device (CCD) sensor, a complementary metal oxide semiconductor (CMOS) sensor, etc., but it is not limited thereto.

The light source 110 of the light source module 100 can be a light-emitting diode. When the light-emitting diode is excited, it emits the light H toward the first polarizing member 200 and the polarization member 560 simultaneously. The light H moving toward the polarization member 560 can pass through the polarization member 560 and the calibration polarizing member 570, and can be received by the sensor 580. In this embodiment, after the sensor 580 receives the light H, it can transfer the measuring result to the automatic power control driver 590, and the automatic power control driver 590 can adjust the emitting of the light source 110 according to the measuring result. Thus, the inspection device 10 in this embodiment can directly adjust the stability of light of the light source module 100.

When the processing module A1 and the processing module A3 include the lock-in amplifier, they can enlarge the small signal and remove the noise, so that the measuring result can be facilitated to obtain.

The features between the aforementioned embodiments can be used or combined as long as they do not violate or conflict the spirit of the present application.

Referring to FIG. 10, an inspection method for inspecting the analyte in the liquid by the aforementioned inspection device is further provided. The step T1 is applied firstly. The step T1 is to provide an inspection device. In an embodiment of the invention, the inspection device can be anyone of the inspection device shown in FIGS. 2A-9B. The components or the structures of the inspection device can be referred to the aforementioned embodiments, so that the features thereof are not repeated in the interest of brevity. The step T2 can be applied secondly. The step T2 is to let a liquid entering the accommodating space. The step T3 can be applied thirdly. The step T3 is to provide a light by a light source module, wherein a portion of the light passes through the first polarizing member, the liquid, and the second polarizing member and reaches the sensing member, and another portion of the light enters the calibration assembly. Finally, the step T4 can be applied. The step T4 is to transfer measuring data from the inspection device to a reading device.

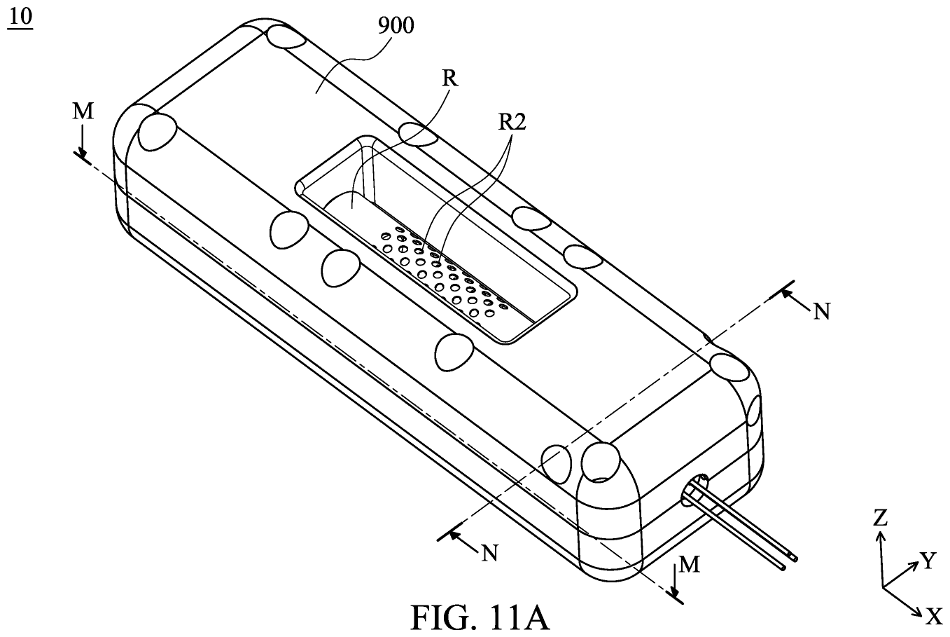

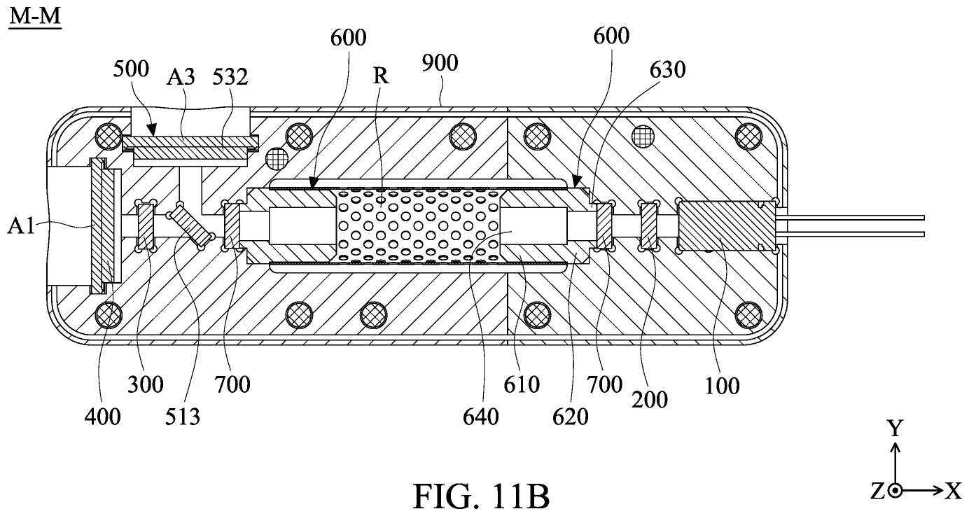

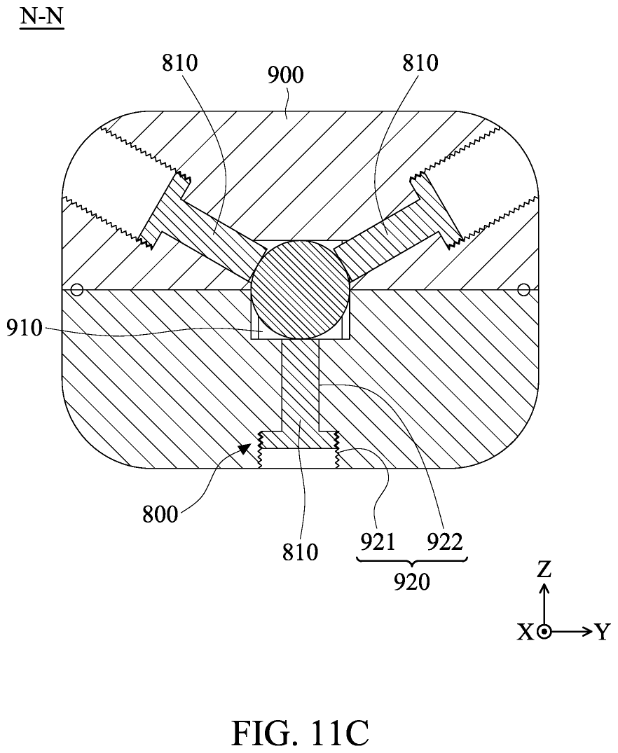

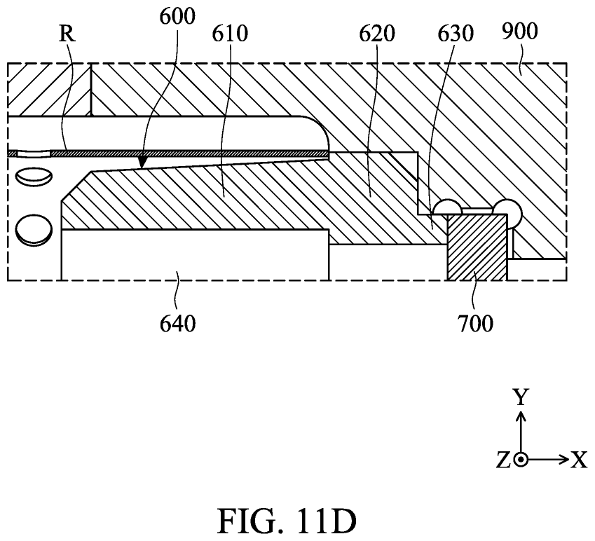

FIG. 11A is a schematic diagram of an inspection device 10 according to another embodiment of the invention, and FIG. 11B and FIG. 11C are cross-sectional views respectively taken along the line M-M and the line N-N in FIG. 11A. As shown in FIG. 11A to FIG. 11C, the inspection device 10 in this embodiment primarily includes a light source module 100, a first polarizing member 200, a second polarizing member 300, a sensing member 400, a calibration assembly 500, and a processing module A1, a hollow carrier R, two plugs 600, two glass sheets 700, and a light path calibration mechanism 800, and the aforementioned components can be accommodated in a case 900 of the inspection device 10. The light source module 100, the first polarizing member 200, the second polarizing member 300, and the sensing member 400, the calibration assembly 500, and the processing module A1 are the same as that shown in the embodiment of FIG. 6, so that the features thereof are not repeated in the interest of brevity. The structure of the hollow carrier R is the same as that shown in the embodiment of FIG. 2B, so that the features thereof are not repeated in the interest of brevity too.

The plugs 600 and the glass sheets 700 are configured to seal the openings on the opposite sides of the hollow carrier R. In detail, each of the plugs 600 can include a positioning portion 610, a sealing portion 620, and an extending portion 630, and the sealing portion 620 can be disposed between the positioning portion 610 and the extending portion 630 and connect the positioning portion 610 to the extending portion 630. The outer diameter of the positioning portion 610 can be less than or equal to the inner diameter of the hollow carrier R. Therefore, when the plug 600 is connected to the hollow carrier R, the positioning portion 610 of the plug 600 can enter the hollow carrier R where does not correspond to the cavity portions R2, and the sealing portion 620 can abut the end of the hollow carrier R. As shown in FIG. 11D, in this embodiment, the positioning portion 610 has a tapered structure (the outer diameter of the portion that is close to the sealing portion 620 is larger than the outer diameter of the portion that is away from the sealing portion 620), so as to seal the opening of the hollow carrier R more tightly.

Referring to FIG. 11A to FIG. 11C, each of the plugs 600 can include a channel 640 penetrating the positioning portion 610, the sealing portion 620, and the extending portion 630, so that the light from the light source module 100 can enter or leave the hollow carrier R through the channel 640. In order to prevent the liquid that enters into the hollow carrier R from the cavity portions R2 flowing out through the channel 640, the glass sheet 700 can be connected to the extending portion 630 of the plug 600 to seal the channel 640. In this embodiment, each of the plugs 600 can include elastic material (such as rubber or silicone). When the glass sheet 700 and the extending portion 630 approach and connect to each other, the extending portion 630 can be slightly deformed. Therefore, there is no gap formed between the glass sheet 700 and the extending portion 630, the outflow of the liquid can be prevented.

In some embodiments, the plugs 600 and the hollow carrier R can be integrally formed in one piece, but it is not limited thereto.

As shown in FIG. 11C, the case 900 can include a space 910 for receiving the light source module 100 and a plurality of guiding slots 920 communicated with the space 910. The light path calibration mechanism 800 includes a plurality of pushing members 810 disposed in different guiding slots 920. In this embodiment, the number of the pushing members 810 takes three as an example (it also can be two or more than three), the angle between the longitudinal axes of the pushing members 810 is 120 degrees, and the calibration of the light path in two dimensions or three dimensions (XYZ) can be processed. When the distance of the at least one pushing member 810 of the light path calibration mechanism 800 entering the guiding slot 920 is adjusted, the pushing member 8100 can abut and push the light source module 100, and the light source module 100 can shift and/or rotate relative to the hollow carrier R. Therefore, the direction of the optical axis of the light from the light source module 100 can be changed to prevent it deviating from the target optical axis, and the light source module 100 can be ensured to emit the light to the desired position.

In this embodiment, each of the guiding slots 920 has a first portion 921 and a second portion 922. The second portion 922 is situated between the space 910 and the first portion 921. In each of the guiding slot 920, screwing thread can be formed on the inner wall of the first portion 920 or the second portion 922. Each of the pushing members 810 can include corresponding screwing thread on the portion that is in contact with the guiding slot 920. When the pushing member 810 rotates, it can adjust that whether the pushing member 810 abut the light source module 100, so that the optical axis thereof can be adjusted to the desired position.

In summary, an embodiment of the invention provides an inspection device and an inspection method. The inspection device is configured to inspect an analyte in a liquid and includes a light source module, a first polarizing member, a second polarizing member, a sensing member, and a calibration assembly. The light source module provides a light, and the first polarizing member is disposed between the light source module and the second polarizing member. An accommodating space for accommodating the aforementioned liquid is formed between the first polarizing member and the second polarizing member. The second polarizing member is disposed between the first polarizing member and the sensing member. When the inspection device inspects the liquid, a portion of the light passes through the first polarizing member, the liquid, and the second polarizing member and reaches the sensing member, and another portion of the light enters the calibration assembly.

Although some embodiments of the present disclosure and their advantages have been described in detail, it should be understood that various changes, substitutions and alterations can be made herein without departing from the spirit and scope of the disclosure as defined by the appended claims. For example, it will be readily understood by those skilled in the art that many of the features, functions, processes, and materials described herein may be varied while remaining within the scope of the present disclosure. Moreover, the scope of the present application is not intended to be limited to the particular embodiments of the process, machine, manufacture, compositions of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. Moreover, the scope of the appended claims should be accorded the broadest interpretation so as to encompass all such modifications and similar arrangements.

While the invention has been described by way of example and in terms of preferred embodiment, it should be understood that the invention is not limited thereto. On the contrary, it is intended to cover various modifications and similar arrangements (as would be apparent to those skilled in the art). Therefore, the scope of the appended claims should be accorded the broadest interpretation to encompass all such modifications and similar arrangements.

What is claimed is:

1. An inspection device for inspecting an analyte in a liquid, comprising:
a light source module, providing a light;
a first polarizing member;
a second polarizing member, wherein the first polarizing member is disposed between the light source module and the second polarizing member;
an accommodating space, disposed between the first polarizing member and the second polarizing member, and configured to accommodate the liquid;
a sensing member, wherein the second polarizing member is disposed between the first polarizing member and the sensing member; and
a calibration assembly, wherein when the light source module emits the light, a portion of the light passes through the first polarizing member, the liquid, and the second polarizing member in sequence and then reaches the sensing member, and another portion of the light enters the calibration assembly.

2. The inspection device as claimed in claim 1, wherein the calibration assembly comprises:
a light splitting member, disposed between the first polarizing member and the accommodating space;
a calibration polarizing member, wherein the polarizing angle of the calibration polarizing member is the same as the polarizing angle of the second polarizing member; and
a calibration sensing member, wherein the calibration polarizing member is disposed between the light splitting member and the calibration sensing member.

3. The inspection device as claimed in claim 2, wherein the calibration assembly further comprises a lock-in amplifier, and the lock-in amplifier is electrically connected to the calibration sensing member.

4. The inspection device as claimed in claim 1, wherein the calibration assembly comprises:
a first light splitting member, disposed between the first polarizing member and the accommodating space;
a calibration polarizing member, wherein the polarizing angle of the calibration polarizing member is the same as the polarizing angle of the second polarizing member;
at least one reflecting member, wherein the calibration polarizing member is disposed between the first light splitting member and the reflecting member; and
a second light splitting member, disposed between the second polarizing member and the sensing member.

5. The inspection device as claimed in claim 4, wherein the first light splitting member guides a portion of the light to the calibration polarizing member, this portion of the light passes through the calibration polarizing member and is reflected to the second splitting member by the reflecting member, and the second splitting member guides this portion of the light to the sensing member.

6. The inspection device as claimed in claim 4, wherein an additional accommodating space is disposed between the reflecting member and the second light splitting member, and the liquid does not enter the additional accommodating space.

7. The inspection device as claimed in claim 1, wherein the calibration assembly comprises:
a first light splitting member, disposed between the first polarizing member and the accommodating space;
a calibration polarizing member, wherein the polarizing angle of the calibration polarizing member is the same as the polarizing angle of the second polarizing member;
a second light splitting member, disposed between the second polarizing member and the sensing member; and
a fiber member, having a first end and a second end, wherein the first end faces the calibration polarizing member, the second end faces the second splitting member, and the calibration polarizing member is disposed between the first end and the first light splitting member.

8. The inspection device as claimed in claim 1, wherein the calibration assembly comprises:
a light splitting member, disposed between the first polarizing member and the accommodating space;
a calibration polarizing member, wherein the polarizing angle of the calibration polarizing member is the same as the polarizing angle of the second polarizing member; and
a fiber member, having a first end and a second end, wherein the first end faces the calibration polarizing member, the second end faces the sensing member, and the calibration polarizing member is disposed between the first end and the light splitting member.

9. The inspection device as claimed in claim 8, wherein the calibration assembly further comprises a first shutter and a second shutter, the first shutter is disposed between the light splitting member and the accommodating space, and the second shutter is disposed between the light splitting member and the calibration polarizing member.

10. The inspection device as claimed in claim 1, wherein the calibration assembly comprises a light splitting member and a calibration sensing member, the light splitting member is disposed between the accommodating space and the second polarizing member, and the light splitting member guides a portion of the light to the calibration sensing member.

11. The inspection device as claimed in claim 10, wherein the calibration assembly further comprises a lock-in amplifier, and the lock-in amplifier is electrically connected to the calibration sensing member.

12. The inspection device as claimed in claim 1, wherein the calibration assembly further comprises a calibration sensing member, and the second polarizing member is disposed between the first polarizing member and the calibration sensing member, wherein the second polarizing member has a first section and a second section, the first section has the function of polarizing, the second section does not have the function of polarizing, and the sensing member and the calibration sensing member respectively correspond to the first section and the second section.

13. The inspection device as claimed in claim 1, wherein the inspection device further comprises a lock-in amplifier, and the lock-in amplifier is electrically connected to the sensing member.

14. The inspection device as claimed in claim 1, wherein the inspection device further comprises a first chiral mirror and a second chiral mirror, the first chiral mirror is disposed between the first polarizing member and the accommodating space, and the second chiral mirror is disposed between the accommodating space and the second polarizing member.

15. The inspection device as claimed in claim 1, wherein the light source module comprises a light source, the calibration assembly is disposed in the light source module and comprises:
a calibration polarizing member;
a sensor;
an automatic power control driver, electrically connected to the sensor and the light source; and
a polarization member, disposed between the light source and the calibration polarizing member, wherein the calibration polarizing member is disposed between the sensor and the polarization member, the polarizing angle of the polarization member is the same as the polarizing angle of the first polarizing member, and the polarizing angle of the calibration polarizing member is the same as the polarizing angle of the second polarizing member.

16. The inspection device as claimed in claim 15, wherein the inspection device further comprises a light path calibration mechanism connected to the light source module to calibrate an optical axis of the light source module.

17. The inspection device as claimed in claim 1, wherein the inspection device further comprises:
a hollow carrier, the accommodating space is formed in the hollow carrier;
a plug, having a channel, wherein the plug is connected to the hollow carrier and seals an opening of the hollow carrier; and
a glass sheet, connected to the plug and sealing the channel.

18. An inspection method, comprising:
providing an inspection device, wherein the inspection device comprises a light source module, a first polarizing member, a second polarizing member, a sensing member, and a calibration assembly, and an accommodating space is formed between the first polarizing member and the second polarizing member;
letting a liquid enter the accommodating space;
using the light source module to provide a light, wherein a portion of the light passes through the first polarizing member, the liquid, and the second polarizing member and reaches the sensing member, and another portion of the light enters the calibration assembly.

19. The inspection method as claimed in claim 18, wherein the calibration assembly comprises:
a light splitting member, disposed between the first polarizing member and the accommodating space;
a calibration polarizing member, wherein the polarizing angle of the calibration polarizing member is the same as the polarizing angle of the second polarizing member; and
a calibration sensing member, wherein the calibration polarizing member is disposed between the light splitting member and the calibration sensing member.

20. The inspection method as claimed in claim 19, wherein the calibration assembly further comprises a lock-in amplifier, and the lock-in amplifier is electrically connected to the calibration sensing member.
